(19) United States
(12) Patent Application Publication
(10) Pub. No.: US 2021/0169371 A1
(43) Pub. Date: Jun. 10, 2021

(54) Cervical Dilation/Dilatation Measurement System of the Uterine Cervix During the Labor of Pregnant Women and Methods of Use

(71) Applicant: SANFORD HEALTH, Sioux Falls, SD (US)

(72) Inventor: Chawki Nahabet, Pensacoda, FL (US)

(21) Appl. No.: 16/760,989

(22) PCT Filed: Nov. 5, 2018
(86) PCT No.: PCT/US2018/059162

§ 371 Date: May 1, 2020

(51) Int. Cl. A61B 5/107; A61B 5/00

(52) U.S. Cl. CPC A61B 5/1076; A61B 5/435; A61B 2560/066; A61B 5/6875; A61B 5/743; A61B 5/6884

(57) ABSTRACT

The present disclosure provides an apparatus including a first clip configured to move between an open position and a closed position. The apparatus also includes a second clip configured to move between an open position and a closed position. The apparatus also includes an elongated connector that is semi-rigid and deformable and that has a first end and a second end. The first end of the elongated connector is coupled to the first clip, and the second end of the elongated connector is coupled to the second clip. The apparatus also includes a sensor coupled to the elongated connector between the first end and the second end.

### CROSS REFERENCE TO RELATED APPLICATIONS

This application claims the benefit of priority to U.S. Provisional Application No. 62/581,152 and PCT Application US2018059162 entitled “Cervical Dilation/Dilatation Measurement System of the Uterine Cervix During the Labor of Pregnant Women and Methods of Use,” filed on Nov. 3, 2017 and Nov. 5, 2018 respectively, the contents of which are hereby incorporated by reference in their entirety.

### BACKGROUND THE INVENTION

Pregnant women routinely undergo multiple pelvic examinations during labor to determine the dilatation of the cervix to monitor the progress of their labor. Current evaluations and measurements of the cervix are done manually by inserting one or both of the index and middle fingers into the vagina of the patient. However, this repeated manual vaginal examination of the cervix is painful/uncomfortable, invasive, subjective, and inaccurate depending on the different medical providers evaluating the cervix and the progress of labor. The dilation of the cervix cannot be measured continuously in a reliable manner if it is done manually.

Further, the traditional manual evaluation to determine the dilatation of the cervix may increase the risk of vaginal infection, post-partum infection, minor and major, including death of the patient. Therefore, a device and corresponding procedure that is less invasive and simultaneously eliminates inaccuracies, variations, human error, and subjectivity of measuring and reporting the dilatation of the cervix during labor is desirable. This device will substantially decrease the associated morbidity and mortality of repeated pelvic examinations.

### SUMMARY OF THE INVENTION

The present disclosure relates to an improved cervical dilation/dilatation measurement system of a uterine cervix of pregnant women during labor and methods of use thereof that enables a medical provider to continuously monitor the dilation/dilatation of the uterine cervix to monitor the progress of their labor.

Thus, in a first aspect, the present invention provides an apparatus consisting of (a) a first clip configured to move between an open position and a closed position, (b) a second clip configured to move between an open position and a closed position, (c) an elongated connector that is semi-rigid and deformable and that has a first end and a second end, where the first end of the elongated connector is coupled to the first clip, and where the second end of the elongated connector is coupled to the second clip, and (d) a sensor coupled to the elongated connector between the first end and the second end.

In a second aspect, the present invention provides a method consisting of (a) attaching the first clip of the apparatus according to the first aspect to a first extent of a cervix of a patient, (b) attaching the second clip of the apparatus according to the first aspect to a second extent of the cervix, where the second extent of the cervix is opposite the first extent of the cervix, and (c) determining, based on a measurement from the sensor, a distance between the first clip and the second clip.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 illustrates an apparatus in a deployment position, according to an example embodiment.

FIG. 2A illustrates an example clip of the apparatus of FIG. 1 in a closed position, according to an example embodiment.

FIG. 2B illustrates an example clip of the apparatus of FIG. 1 in an open position, according to an example embodiment.

FIG. 3 illustrates an example actuator of the apparatus of FIG. 1, according to an example embodiment.

FIG. 4 illustrates the apparatus of FIG. 1 in a deployed position, according to an example embodiment.

FIG. 5 illustrates another example apparatus in a deployment position, according to an example embodiment.

FIG. 6 illustrates the apparatus of FIG. 1 in a deployed position at a first time, in accordance with one embodiment of the invention.

FIG. 7 illustrates the apparatus of FIG. 1 in the deployed position at a second time, in accordance with one embodiment of the invention.

FIG. 8 is a schematic drawing of a computer network infrastructure, according to an example embodiment.

FIG. 9 is a flow chart depicting functions carried out in accordance with example embodiments of the disclosed methods.

### DETAILED DESCRIPTION OF THE INVENTION

Exemplary devices and methods are described herein. It should be understood that the word “exemplary” is used herein to mean “serving as an example, instance, or illustration.” Any embodiment or feature described herein as “exemplary” is not necessarily to be construed as preferred or advantageous over other embodiments or features. The exemplary embodiments described herein are not meant to be limiting. One of ordinary skill in the art will readily understand that certain aspects of the disclosed systems and methods can be arranged and combined in a wide variety of different configurations, all of which are contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an exemplary embodiment may include elements that are not illustrated in the Figures.

As used herein, with respect to measurements, “about” means +/−5%.

As used herein, “semi-rigid” means a structure that is stiff and solid to maintain its shape in the absence of an external force, but not inflexible such that it can change shape in response to an external force.

As used herein, “deformable” means a structure that can change its shape while being acted upon by any kind of external force.

As used herein, a “catheter” is an apparatus that is connected to a deployment mechanism and houses a medical device. The catheter may be used for delivering the apparatus described herein to the cervix. A catheter can have braided metal strands within the catheter wall to maintain structural integrity. The structural elements of the tip of the catheter can be bonded or laser welded to the braided strands of the catheter to improve the performance characteristics of the catheter tip.

As used herein, “lumen” refers to a passage in a tubular housing or a catheter through which a medical device may be disposed.

As used herein, “dilation” and “dilatation” may be used interchangeably to describe the size of an opening of a uterine cervix of a pregnant woman in labor.

As used herein, “electrical connection” means a wired connection, wireless connection, optical connection, or any other connection between two components such that the two components can share data between each other.

As used herein, “adjacent to the cervix in the vagina” means in the vicinity of the cervix in the vagina, nearby the cervix in the vagina, or close to the cervix in the vagina.

As used herein, “extent of the cervix” refers to an edge of the external orifice of the cervix. A distance between opposing extents of the cervix define a degree of cervical dilatation/dilation.

As used herein, “medical provider” means a doctor of medicine (M.D.), a registered nurse (R.N.), a certified nurse-midwife (C.N.M.), a medical student, mid-wife or any other provider.

As used herein, “coupled” means associated directly as well as indirectly. For example, a member A may be directly associated with a member B, or may be indirectly associated therewith, e.g., via another member C. It will be understood that not all relationships among the various disclosed elements are necessarily represented.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, a system, apparatus, device, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

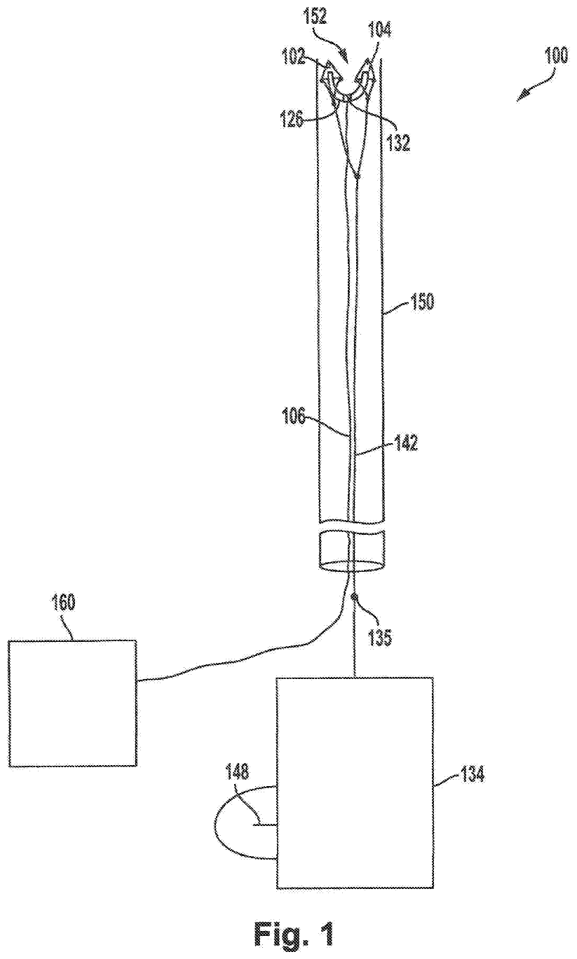

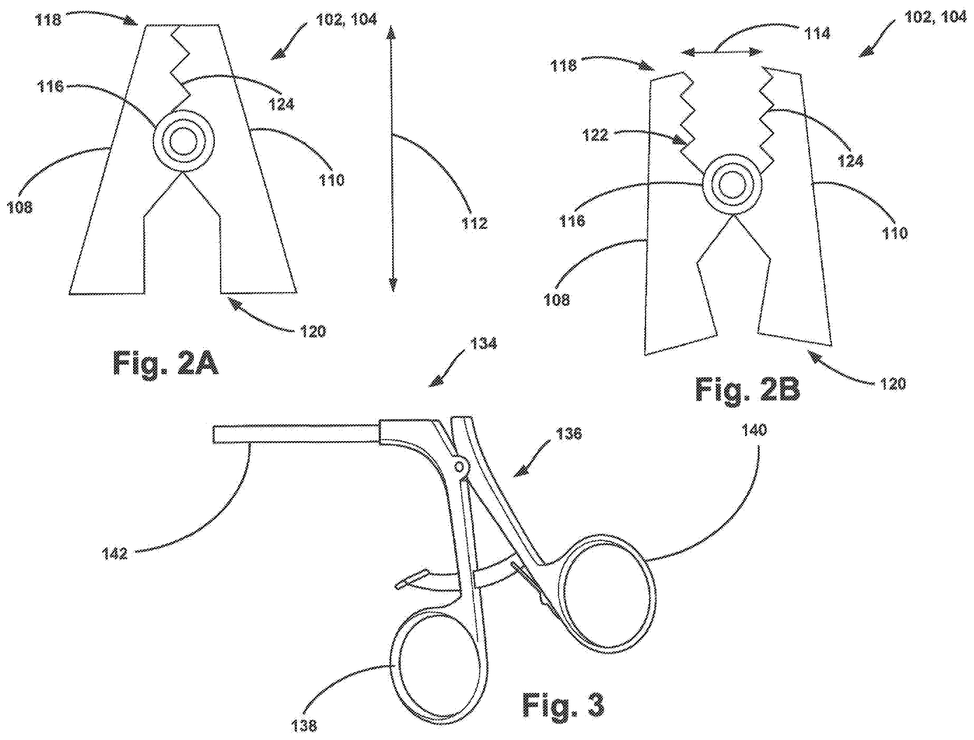

With reference to the Figures, FIG. 1 illustrates an example apparatus 100 including a first clip 102 configured to move between an open position and a closed position, and a second clip 104 configured to move between an open position and a closed position. FIGS. 2A-2B illustrates an example clip, according to an example embodiment. Both the first clip 102 and the second clip 104 may have a similar structure to the clip shown in FIGS. 2A-2B. Each of the first clip 102 and the second clip 104 have a pair of gripping arms 108, 110 that have a gripping length 112 ranging from about 1 mm to about 3 cm or any other appropriate length, and a gripping width 114 ranging from about 1 mm to about 3 cm or any other appropriate width.

In one example, the first clip 102 and the second clip 104 may each include a pair of gripping arms 108, 110 that are biased to the closed position via a spring 116.

The first and second clips 102, 104 may have two ends, a gripping end 118 and a pinching end 120. Such a spring 116 may be a torsion spring positioned between the gripping end 118 and the pinching end 120 of the first and second clips 102, 104. The pinching end 120 of the first and second clips 102, 104 may be spaced apart when the first and second clips 102, 104 are in the closed position. The first and second clips 102, 104 may be transitioned from the closed position (shown in Figured 2A) to the open position (shown in FIG. 2B) by squeezing the pair of gripping arms 108, 110 together at the pinching end 120. In such an example, the first and second clips 102, 104 may be sized such that the first and second clips 102, 104 extend from the cervix in the vagina to outside of the vagina, such that a medical provider could open and close the first and second clips 102, 104 manually from outside of the vagina.

In one example, an inner surface 122 of each of the first clip 102 and the second clip 104 is substantially smooth. In another example, an inner surface 122 of each of the first clip 102 and the second clip 104 may include ridges or teeth 124 to grip an extent of the cervix that is positioned between the gripping arms 108, 110 of the first and second clips 102, 104.

The ridges or teeth 124 may comprise a different material than the gripping arms 108, 110 of the first and second clips 102, 104. For example, the ridges or teeth 124 may comprise a biocompatible material. In another example, the ridges or teeth 124 may be the same material as the gripping arms 108, 110 of the first and second clips 102, 104. Other configurations are possible as well.

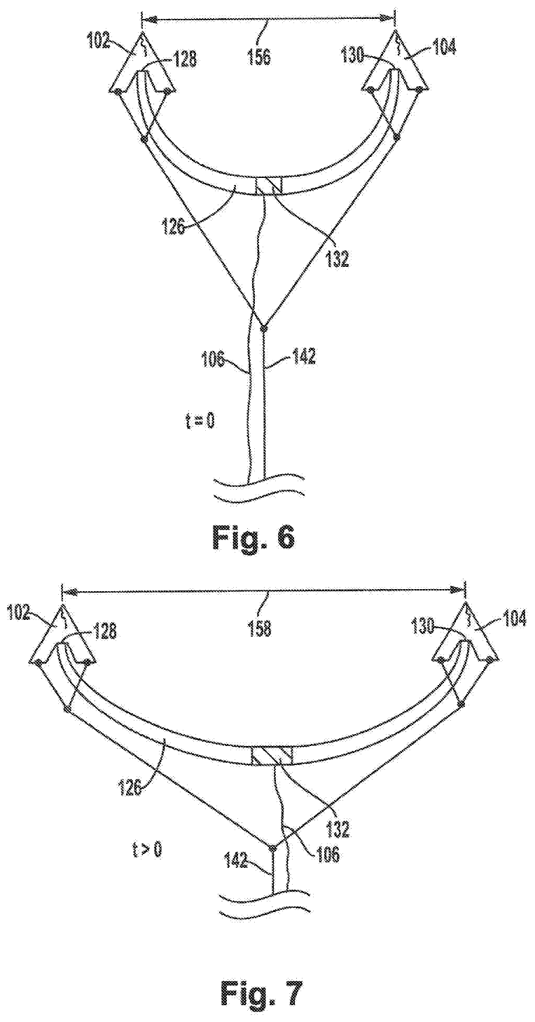

The apparatus 100 may further include an elongated connector 126 that is semi-rigid and deformable and that has a first end 128 and a second end 130, as more clearly shown in FIGS. 6 and 7. The deformable property of the elongated connector 126 enables the elongated connector 126 to expand and change shape after it is connected to the cervix.

As shown in FIG. 1, the first end 128 of the elongated connector 126 is coupled to the first clip 102, and the second end 130 of the elongated connector 126 is coupled to the second clip 104. The apparatus 100 may further include a sensor 132 coupled to the elongated connector 126 between the first end 128 and the second end 130. The sensor 132 may be positioned on an exterior surface of the elongated connector, or may be embedded in the elongated connector 126. The semi-rigid property of the elongated connector 126 enables the elongated connector 126 to have an unstressed length and shape that is known, and this known length and shape of the unstressed elongated connector 126 enables a “zeroing” of the sensor 132 prior to or just after attachment of the apparatus to the cervix. In one example, the sensor 132 may be a linear displacement sensor. In another example, the sensor 132 may be an angular displacement sensor. As other examples, the sensor 132 may include magnetoresistive (MR) sensors, fluxgate magnetometers, or Hall effect sensors. Other examples are possible as well.

As shown in FIG. 1, the apparatus 100 may further include an applicator or actuator 134 coupled to the first clip 102 and the second clip 104. The actuator 134 may be configured to move the first clip 102 and the second clip 104 between the open position and the closed position. The actuator 134 need not be directly attached to the first and second clips 102, 104, but may be connected in an indirect manner with other elements in between them. The actuator 134 may be any mechanism capable of moving the first and second clips 102, 104 between the open and closed positions.

In one example, shown in FIG. 3, the actuator 134 may include a handle 136 for grasping by a medical provider, including a stationary arm 138 and a lever arm 140. The lever arm 140 may be coupled to a rod 142, which extends from the handle 136 to the first and second clips 102, 104 and is coupled to the first and second clips 102, 104 at the distal end of the rod 142. Squeezing the handle 136 of the actuator 134 closed causes the rod 142 to slide in the proximal direction and causes the first and second clips 102, 104 to close. Opening the handle 136 causes the first and second clips 102, 104 to open. The handle 136 may also be a ratcheting handle, as shown in FIG. 3, to lock the handle 136, and the first and second clips 102, 104 in place.

In further examples, the handle 136 of the actuator 134 may be spring biased, such that the first and second clips 102, 104 are biased in a closed position. Other configurations and examples of the actuator 134 are contemplated. The actuator 134 may be removably coupled to the apparatus 100 via a connector mechanism 135, such that once the apparatus 100 is deployed, the actuator 134 can be decoupled from the apparatus 100. Using the example described above, the handle 136 may be removably coupled from the rod 142 of the actuator 134. For example, the handle 136 may include a threaded connection with the rod 142, such that the two components can be unscrewed to detach them from one another. In another example, the handle 136 may be removably press fit into the rod 142 of the actuator 134. Other connector mechanisms 135 are possible as well. The arrangement of a removable actuator 134 provides additional comfort and maneuverability of the patient after coupling the first and second clips 102, 104 to the cervix of the patient.

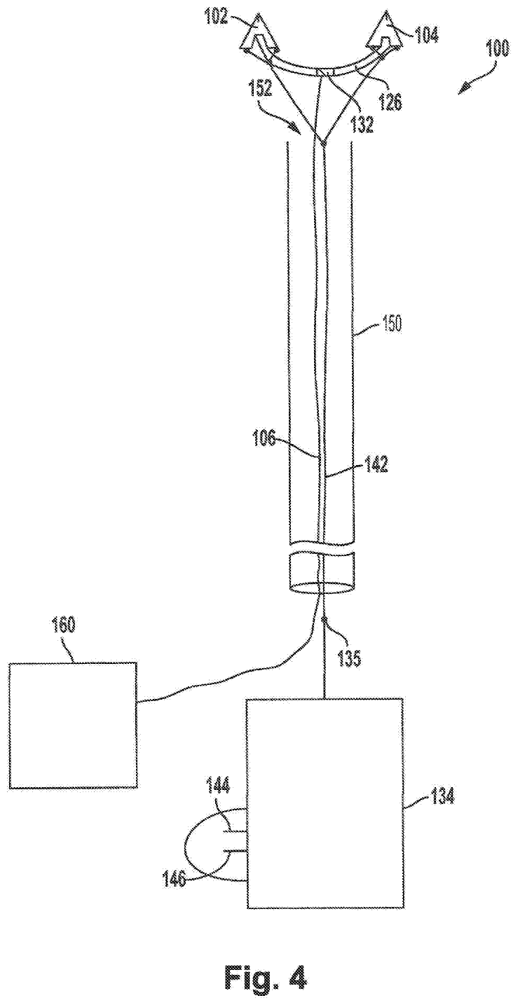

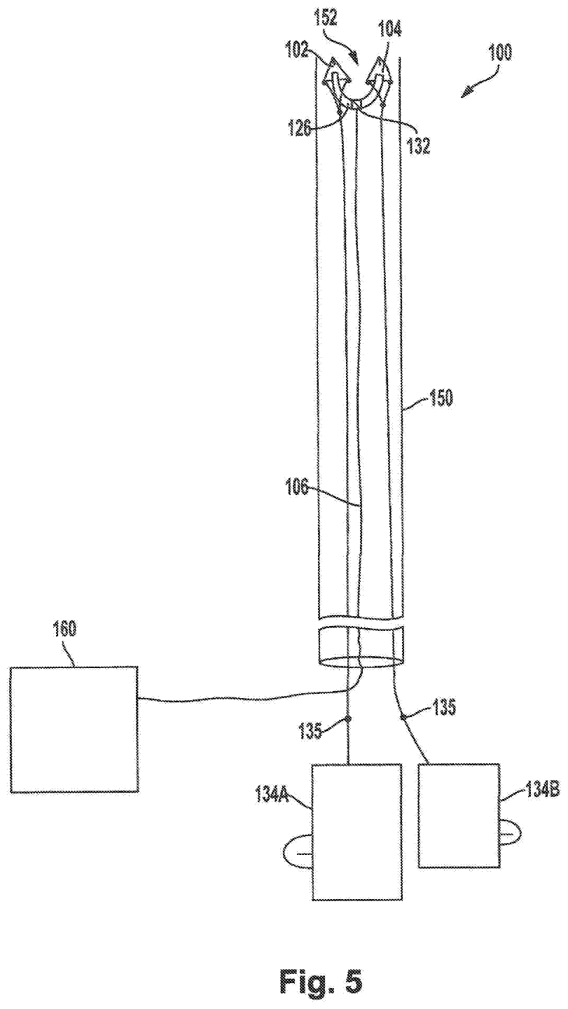

In one example, as shown in FIG. 1, a single trigger mechanism 148 is configured to move both the first clip 102 and the second clip 104 between the open position and the closed position, either simultaneously or in succession. In such an example, the single trigger mechanism 148 may comprise a single handle, a single lever, or a single button. In another example, as shown in FIG. 4, the actuator 134 comprises a first trigger mechanism 144 configured to move the first clip 102 between the open position and the closed position, and the actuator 134 further comprises a second trigger mechanism 146 configured to move the second clip 104 between the open position and the closed position. In one example, the first and second trigger mechanisms 144, 146 may include two separate handles. In another example, the first and second trigger mechanisms 144, 146 may include two levers (as shown in FIG. 4) or two separate buttons.

In yet another example, as shown in FIG. 5, the apparatus 100 includes a first actuator 134A coupled to the first clip 102 and a second actuator 134B coupled to the second clip 104. In such an example, the first actuator 134A is configured to move the first clip 102 between the open position and the closed position, and the second actuator 134B is configured to move the second clip between the open position and the closed position, either simultaneously or in succession. In such an example, both the first actuator 134A and the second actuator 134B may be removably coupled to the apparatus 100, as discussed above. In one example, each of the first actuator 134A and the second actuator 134B may be similarly arranged to the handle 136 described above in relation to FIG. 3.

As shown in FIG. 1, the apparatus 100 may further include an introducer catheter 150 having a lumen 152. In such an example, each of the first clip 102, the second clip 104, the elongated connector 126, and the sensor 132 are positioned at least partially within the lumen 152 of the introducer catheter 150 in a deployment position. The introducer catheter 150 may have a diameter ranging from about 1 mm to about 2 cm, and preferably between about 4 mm and about 10 mm, for example only and not by way of limitation.

The introducer catheter 150 may be straight, curved, or angled, as examples. As shown in FIG. 4, in use the apparatus 100 may transition from the deployment position to a deployed position such that each of the first clip 102, the second clip 104, the elongated connector 126, and the sensor 132 are positioned outside of the lumen 152 of the introducer catheter 150 in the deployed position. Such a position may be achieved by retracting the introducer catheter 150 with respect to the first clip 102 and the second clip 104, or by advancing the first clip 102 and the second clip 104 with respect to the introducer catheter 150.

The elongated connector 126 may take various forms, including a cable, wire, band, or ribbon as examples. In one example, the elongated connector 126 comprises a shape memory material, for example, nitinol (nickel-titanium), titanium, titanium alloys, copper-aluminum-nickel alloys, various plastics, or any other suitable material capable of retaining shape memory. The shape memory material of the semi-rigid deformable elongated connector 126 may be biased to have a U-shape.

As shown in FIG. 6, the semi-rigid deformable elongated connector 126 at time (t) has a natural, unstressed U-shape such that there is a distance 156 between the first clip and the second clip. Time (t) is either before attaching the apparatus 100 to a cervix of a patient or immediately after attaching the apparatus to the cervix of the patient. The sensor 132 may be “zeroed” at time (t) as the semi-rigid deformable elongated connector 126 may have a known distance 156 at time (t) prior to attaching the apparatus to a cervix of a patient.

In one example, the distance 156 is approximately 2 cm. Such a distance 156 may correspond to a cervix that is not dilated, or is just beginning to dilate during labor. FIG. 7 illustrates elongated connector 126 at a second point in time (t>0). At time t>0, the cervix has dilated such that the distance 158 between the first clip 102 and the second clip 104 is greater than the initial distance 156.

The apparatus 100 may further include a processor 160 in communication with the sensor 132, as shown in FIGS. 1 and 4. The processor 160 may be configured to determine the distance between the first clip 102 and the second clip 104 based on a reading from the sensor 132. In particular, the processor 160 may receive a change in resistance or other change in property of the sensor 132 based on the change in distance between the first clip 102 and the second clip 104 that causes the elongated connector 126 to change its shape, and may be configured to determine the distance between the first clip 102 and the second clip 104 based on that change in resistance. In operation, as the first and second clips 102, 104 are attached to opposite sides of the cervix when in use, the measured distance between the first clip 102 and the second clip 104 is an indication of a dilation of a uterine cervix of a pregnant woman in labor.

The apparatus 100 may be configured to monitor this process continuously, or may measure the distance between the first clip 102 and the second clip 104 periodically, such as every minute, every five minutes, or every ten minutes as examples. In one example, the processor 160 is in communication with the sensor 132 of the apparatus 100 via an electrical connection 106. Such an electrical connection 106 may be a wired connection (as shown in FIGS. 1 and 4), a wireless connection, an optical connection, or any other connection between the sensor 132 and the processor 160 such that the sensor 132 and processor 160 can share data between each other. In the case where the electrical connection 106 is a wired connection, the wire may be secured to the leg of the patient with a holder to prevent dislodgement or displacement of the apparatus 100 during labor.

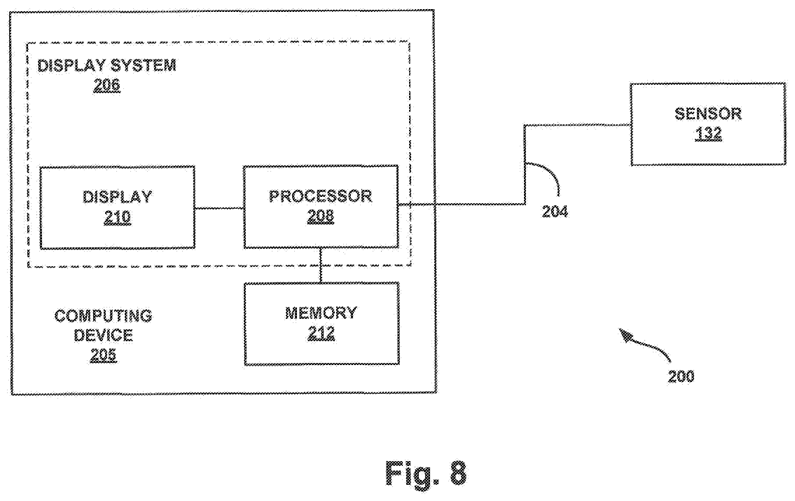

FIG. 8 illustrates an example schematic drawing of a computer network infrastructure that may be in communication with one or more components of the apparatus 100 described above. In one system 200, a computing device 205 communicates with the sensor 132 of the apparatus 100 using a communication link 204, such as a wired or wireless connection. The computing device 202 may be any type of device that can receive data and display information corresponding to or associated with the data. For example, the computing device 202 may be a mobile phone, a tablet, or a personal computer as examples.

Thus, the computing device 202 may include a display system 206 comprising a processor 208 and a display 210. The display 210 may be, for example, an optical see-through display, an optical see-around display, or a video see-through display. The processor 208 may receive data from the sensor 132, and configure the data for display on the display 210. For example, the display 210 may provide a graph illustrating the dilation of the cervix of the patient on the y-axis, with time on the x-axis. Such a graph may be printed in paper for review by the medical provider. Depending on the desired configuration, processor 208 can be any type of processor including, but not limited to, a microprocessor, a microcontroller, a digital signal processor, or any combination thereof.

The computing device 202 may further include on-board data storage, such as memory 212 coupled to the processor 208. The memory 212 may store software that can be accessed and executed by the processor 208, for example. The memory 212 can include any type of memory now known or later developed including but not limited to volatile memory (such as RAM), non-volatile memory (such as ROM, flash memory, etc.) or any combination thereof.

According to an example embodiment, the computing device 202 may include program instructions that are stored in the memory 212 (and/or possibly in another data-storage medium) and executable by the processor 208 to facilitate the various functions described herein. Although various components of the system 200 are shown as distributed components, it should be understood that any of such components may be physically integrated and/or distributed according to the desired configuration of the computing system.

The sensor 132 and the computing device 202 may contain hardware to enable the communication link 204, such as processors, transmitters, receivers, antennas, etc.

In FIG. 8, the communication link 204 is illustrated as a wired connection; however, a wireless connection may also be used. For example, the communication link 204 may be a wired link via a serial bus such as a universal serial bus or a parallel bus. A wired connection may be a proprietary connection as well. The communication link 204 may also be a wireless connection using, e.g., Bluetooth® radio technology, communication protocols described in IEEE 802.11 (including any IEEE 802.11 revisions), Cellular technology (such as GSM, CDMA, UMTS, EV-DO, WiMAX, or LTE), or Zigbee® technology, among other possibilities.

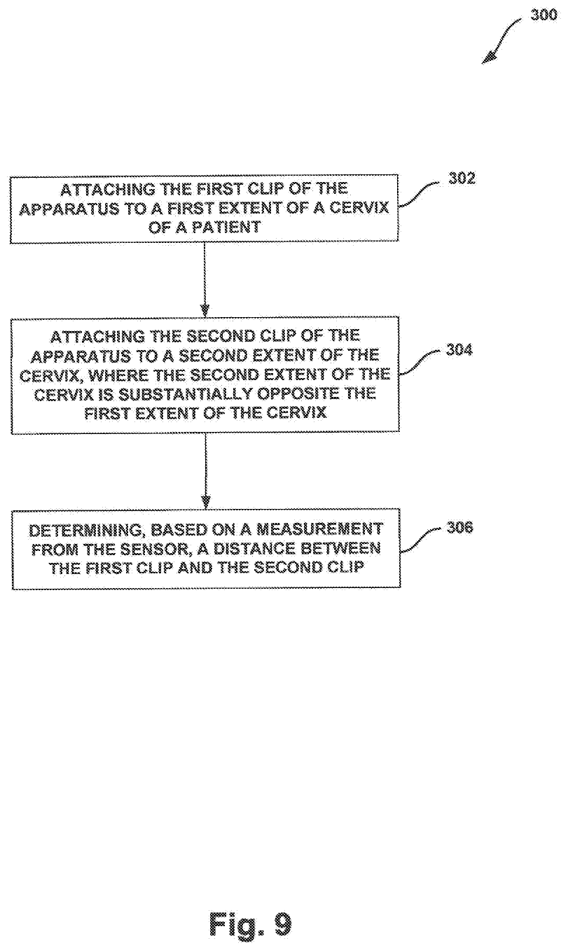

FIG. 9 is a block diagram of an example method for measuring cervical dilation/dilatation of the uterine cervix during the labor of pregnant women. Method 300 shown in FIG. 9 presents an embodiment of a method that could be used by the apparatus 100 of FIGS. 1-6, as described above for example only and not by limitation. Method 300 may include one or more operations, functions, or actions as illustrated by one or more of blocks 302-306. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

Initially, at block 302, the method 300 includes attaching the first clip 102 of the apparatus 100 as described above in relation to FIGS. 1-6 to a first extent of a cervix. The method 300 continues with attaching the second clip 104 of the apparatus 100 as described above in relation to FIGS. 1-6 to a second extent of the cervix, where the second extent of the cervix is opposite the first extent of the cervix. As the cervix is approximately circular in shape, positioning the first clip 102 and the second clip 104 on opposite extents of the cervix means that the first clip 102 and the second clip 104 are positioned approximately one hundred-eighty degrees apart. Such an arrangement helps measure the maximum dilation of the cervix. The method 300 continues with determining, based on a measurement from the sensor 132, a distance between the first clip 102 and the second clip 104.

In one example, attaching the first clip 102 to the first extent of the cervix includes (i) transitioning the first clip 102 from the closed position to the open position, (ii) positioning a portion of the first extent of the cervix between two gripping arms 108, 110 of the first clip 102, and (iii) transitioning the first clip 102 to the closed position to thereby attach the first clip 102 to the first extent of the cervix, and attaching the second clip 104 to the second extent of the cervix includes (i) transitioning the second clip 104 from the closed position to the open position, (ii) positioning a portion of the second extent of the cervix between two gripping arms 108, 110 of the second clip, and (iii) transitioning the second clip 104 to the closed position to thereby attach the second clip 104 to the second extent of the cervix.

In one example, as described above, the apparatus 100 further includes an actuator 134 coupled to the first clip 102 and the second clip 104. In such an example, transitioning the first clip 102 from the closed position to the open position includes actuating the actuator 134 in response to a first signal, and transitioning the second clip 104 from the closed position to the open position includes actuating the actuator 134 in response to the first signal or a second signal. In one example, the first signal includes a first gripping of the handle 136 of the actuator 134 by a user as described above in relation to FIG. 3. In such an example, the second signal includes a second gripping of the handle 136 by the user. In another example, the first signal includes a wireless signal/electrical signal generated by a processor 160 that wirelessly actuates the first clip 102, and the second signal includes a wireless signal/electrical signal generated by the processor 160 that wirelessly actuates the second clip 104. The actuator 134 may be removably coupled to the apparatus 100, such that once the first clip 102 is attached to the first extent of the cervix and the second clip 104 is attached to the second extent of the cervix, the actuator 134 can be decoupled from the apparatus 100. As discussed above, such an arrangement provides additional comfort and maneuverability of the patient.

In yet another example, the apparatus 100 includes a first actuator 134A coupled to the first clip 102 and a second actuator 134B coupled to the second clip 104. In such an example, transitioning the first clip 102 from the closed position to the open position includes actuating the first actuator 134A in response to a first signal, and transitioning the second clip 104 from the closed position to the open position includes actuating the second actuator 134B in response to a second signal. In such an example, both the first actuator 134A and the second actuator 134B may be removably coupled to the apparatus, such that once the first clip 102 is attached to the first extent of the cervix and the second clip 104 is attached to the second extent of the cervix, the first actuator 134A and the second actuator 134B can be decoupled from the apparatus 100.

In one example, the method 300 further includes (i) transitioning the first clip 102 to the open position to thereby detach the first clip 102 from the first extent of the cervix, and (ii) transitioning the second clip 104 to the open position to thereby detach the second clip 104 from the second extent of the cervix. Once the apparatus 100 is detached from the cervix, the apparatus can be removed from the vagina of the patient.

In another example, the method 300 further includes, prior to attaching the first clip 102 and the second clip 104 to the cervix, introducing an introducer catheter 150 adjacent to the cervix in the vagina. The introducer catheter 150 includes a lumen 152, and each of the first clip 102, the second clip 104, the elongated connector 126, and the sensor 132 are positioned at least partially within a lumen 152 of the introducer catheter 150 in a deployment position and arranged in a U-shape.

In one example, the medical provider may position the introducer catheter 150 between their index and middle fingers. Then the medical provider may guide or position the introducer catheter 150 adjacent to the cervix in the vagina. Next the medical provider may retract the introducer catheter 150 with respect to the first clip 102 and the second clip 104 such that each of the first clip 102, the second clip 104, the elongated connector 126, and the sensor 132 are positioned outside of the lumen 152 of the introducer catheter 150 in a deployed position. The medical provide may then guide the first clip 102 to one edge of the cervix to attach the first clip 102 thereto and guide the second clip 104 to an opposing edge of the cervix to attach the second clip 104 thereto.

In another example, the method 300 further includes, prior to attaching the first clip 102 and the second clip 104 to the cervix, inserting a vaginal speculum into the vagina of the patient. In such an example, the medical provider may visualize the cervix, and then under direct vision attach the first clip 102 to the first extent of the cervix and attach the second clip 104 to the second extent of the cervix. The vaginal speculum can then be removed from the patient's vagina. Such an example may be used without the introducer catheter 150 at all. In one such example, as discussed above, the first clip 102 and the second clip 104 may each include a pair of gripping arms 108, 110 that are biased to the closed position via a spring 116. Such a spring 116 may be a torsion spring positioned between a gripping end 118 and a pinching end 120 of the first and second clips 102, 104. The first and second clips may be transitioned from the closed position to the open position by squeezing the pair of gripping arms 108, 110 together at the pinching end 120.

In one such example, the first and second clips 102, 104 may be sized such that the first and second clips 102, 104 extend from the cervix in the vagina to outside of the vagina, such that a medical provider could open and close the first and second clips 102, 104 manually from outside of the vagina. As such, with the cervix visible to the medical provider via the vaginal speculum, the medical provider may simply squeeze the pinching end 120 of the first clip 102 to open the gripping arms 108, 110 of the first clip 102, position the first clip 102 adjacent to the first extent of the cervix, release the pinching end 120 of the first clip 102 to close the gripping arms 108, 110 around the first extent of the cervix, and then repeat the process for the second clip 104 to attach the second clip 104 to the second extent of the cervix.

In another example, the medical provider may use both the vaginal speculum and the introducer catheter 150. In such an example, the medical professional may insert the vaginal speculum into the vagina of the patient and open the speculum to visualize the cervix. Then the medical professional may insert the introducer catheter 150 adjacent to the cervix in the vagina, as discussed above.

In the embodiments including the introducer catheter 150, the method 300 may further include (i) retracting the introducer catheter 150 with respect to the first clip 102 and the second clip 104 such that each of the first clip 102, the second clip 104, the elongated connector 126, and the sensor 132 are positioned outside of the lumen 152 of the introducer catheter 150 in a deployed position in a vagina of a patient, (ii) transitioning the first clip 102 to the open position to thereby detach the first clip 102 from the first extent of the cervix, (iii) transitioning the second clip 104 to the open position to thereby detach the second clip 104 from the second extent of the cervix, (iv) advancing the introducer catheter 150 with respect to the first clip 102 and the second clip 104 such that each of the first clip 102, the second clip 104, the elongated connector 126, and the sensor 132 are positioned at least partially inside of the lumen 152 of the introducer catheter 150, and (v) removing the introducer catheter 150 from the vagina of the patient.

The method 300 may further include (i) providing a display of a graph of the distance between the first clip 102 and the second clip 104 over time, and (ii) in response to the graph of the distance between the first clip 102 and the second clip 104, determining and measuring a degree of cervical dilatation/dilation in relation to an amount of time elapsed. Such a display may provide valuable information of continuous monitoring of the degree of cervical dilatation/dilation on one axis of a graph with elapsed time on another axis of the graph, to thereby indicate the progress of the patient's labor.

Certainly, other medical devices such ultrasound technology may be used in association with the present invention in order effectuate the purposes of the invention described herein.

It will be appreciated that other arrangements are possible as well, including some arrangements that involve more or fewer steps than those described above, or steps in a different order than those described above.

While various aspects and embodiments have been disclosed herein, other aspects and embodiments will be apparent to those skilled in the art. All embodiments within and between different aspects of the invention can be combined unless the context clearly dictates otherwise. The various aspects and embodiments disclosed herein are for purposes of illustration and are not intended to be limiting, with the true scope and spirit being indicated by the following claims.

1. An apparatus comprising:
a first clip configured to move between an open position and a closed position;
a second clip configured to move between an open position and a closed position;
an elongated connector that is semi-rigid and deformable and that has a first end and a second end, wherein the first end of the elongated connector is coupled to the first clip, and wherein the second end of the elongated connector is coupled to the second clip; and
a sensor coupled to the elongated connector between the first end and the second end.

2. The apparatus of claim 1, further comprising an actuator coupled to the first clip and the second clip, wherein the actuator is configured to move the first clip and the second clip between the open position and the closed position.

3. The apparatus of claim 2, wherein the actuator is removably coupled to the apparatus.

4. The apparatus of claim 2 wherein the actuator comprises a first trigger mechanism configured to move the first clip between the open position and the closed position, and wherein the actuator further comprises a second trigger mechanism configured to move the second clip between the open position and the closed position.

5. The apparatus of claim 1, further comprising a first actuator coupled to the first clip and a second actuator coupled to the second clip, wherein the first actuator is configured to move the first clip between the open position and the closed position, and wherein the second actuator is configured to move the second clip between the open position and the closed position.

6. The apparatus of claim 5, wherein each of the first actuator and the second actuator are removably coupled to the apparatus.

7. The apparatus of claim 1, further comprising an introducer catheter having a lumen, wherein each of the first clip, the second clip, the elongated connector, and the sensor are positioned at least partially within the lumen of the introducer catheter in a deployment position.

8. The apparatus claim 1, wherein the elongated connector comprises a shape memory material.

9. (canceled)

10. (canceled)

11. The apparatus claim 1, further comprising a processor in communication with the sensor, wherein the processor is configured to determine a distance between the first clip and the second clip based on a reading from the sensor.

12. The apparatus of claim 11, wherein the distance between the first clip and the second clip is an indication of a dilation of a uterine cervix.

13. (canceled)

14. (canceled)

15. (canceled)

16. (canceled)

17. A method comprising:
attaching the first clip of the apparatus according to claim 1 to a first extent of a cervix of a patient;
attaching the second clip of the apparatus according to claim 1 to a second extent of the cervix, wherein the second extent of the cervix is opposite the first extent of the cervix;
determining, based on a measurement from the sensor, a distance between the first clip and the second clip; and
an actuator coupled to the first clip and the second clip, wherein the actuator is configured to move the first clip and the second clip between the open position and the closed position.

18. The method of claim 17, wherein attaching the first clip to the first extent of the cervix comprises (i) transitioning the first clip from the closed position to the open position, (ii) positioning a portion of the first extent of the cervix between two gripping arms of the first clip, and (iii) transitioning the first clip to the closed position to thereby attach the first clip to the first extent of the cervix, and wherein attaching the second clip to the second extent of the cervix comprises (i) transitioning the second clip from the closed position to the open position, (ii) positioning a portion of the second extent of the cervix between two gripping arms of the second clip, and (iii) transitioning the second clip to the closed position to thereby attach the second clip to the second extent of the cervix.

19. (canceled)

20. The method of claim 18, wherein the apparatus further comprises a first actuator coupled to the first clip and a second actuator coupled to the second clip, wherein transitioning the first clip from the closed position to the open position comprises actuating the first actuator in response to a first signal, and wherein transitioning the second clip from the closed position to the open position comprises actuating the second actuator in response to a second signal.

21. (canceled)

22. The method of claim 17 further comprising, prior to attaching the first clip and the second clip to the cervix, introducing an introducer catheter adjacent to the cervix in a vagina of the patient wherein the introducer catheter includes a single lumen, and wherein each of the first clip, the second clip, the elongated connector, and the sensor are positioned at least partially within a lumen of the introducer catheter in a deployment position and arranged in a U-shape.

23. (canceled)

24. The method of claim 22 further comprising:
retracting the introducer catheter with respect to the first clip and the second clip such that each of the first clip the second clip, the elongated connector, and the sensor are positioned outside of the lumen of the introducer catheter in a deployed position in the vagina of the patient;
transitioning the first clip to the open position to thereby detach the first clip from the first extent of the cervix;
transitioning the second clip to the open position to thereby detach the second clip from the second extent of the cervix;
advancing the introducer catheter with respect to the first clip and the second clip such that each of the first clip, the second clip, the elongated connector, and the sensor are positioned at least partially inside of the lumen of the introducer catheter; and
removing the introducer catheter from the vagina of the patient.

25. (canceled)

26. The method of claim 17 further comprising:
providing a display of a graph of the distance between the first clip and the second clip over time; and
in response to the graph of the distance between the first clip and the second clip, determining and measuring a degree of cervical dilatation/dilation in relation to an amount of time elapsed.

27. An apparatus comprising:
a first clip configured to move between an open position and a closed position;
a second clip configured to move between an open position and a closed position;
an elongated connector that is semi-rigid and deformable and that has a first end and a second end, wherein the first end of the elongated connector is coupled to the first clip, and wherein the second end of the elongated connector is coupled to the second clip;
an actuator coupled to the first clip and the second clip, wherein the actuator is configured to move the first clip and the second clip between the open position and the closed position;
a sensor coupled to the elongated connector between the first end and the second end; and
a processor in communication with the sensor, wherein the processor is configured to determine a distance between the first clip and the second clip based on a reading from the sensor.

28. The apparatus of claim 27, further comprising an introducer catheter having a lumen, wherein each of the first clip, the second clip, the elongated connector, and the sensor are positioned at least partially within the lumen of the introducer catheter in a deployment position.

29. The apparatus of claim 27 wherein the actuator comprises a first trigger mechanism configured to move the first clip between the open position and the closed position, and wherein the actuator further comprises a second trigger mechanism configured to move the second clip between the open position and the closed position.

30. The apparatus of claim 27, further comprising a first actuator coupled to the first clip and a second actuator coupled to the second clip, wherein the first actuator is configured to move the first clip between the open position and the closed position, and wherein the second actuator is configured to move the second clip between the open position and the closed position.
